(19) United States
(12) Patent Application Publication
(10) Pub. No.: US 2014/0031310 A1
(43) Pub. Date: Jan. 30, 2014

(54) PHARMACEUTICAL COMPOSITIONS AND METHODS FOR MAKING AND USING THEM

(71) Applicants: John Maki, Mendham, NJ (US); Newell Bascomb, Chester Springs, PA (US); Timothy J. Turner, Belmont, MA (US); Fredric Young, Los Altos, CA (US)

(72) Inventors: John Maki, Mendham, NJ (US); Newell Bascomb, Chester Springs, PA (US); Timothy J. Turner, Belmont, MA (US); Fredric Young, Los Altos, CA (US)

(21) Appl. No.: 13/979,362

(22) PCT Filed: Jan. 18, 2012
(86) PCT No.: PCT/US12/21724

§ 371 Date: Sep. 26, 2013

Related U.S. Application Data
(60) Provisional application No. 61/433,885, filed on Jan. 18, 2011.

(51) Int. Cl. A61K 31/7068; A61K 31/4985; A61K 31/4439; A61K 31/337; A61K 31/44; A61K 31/404; A61K 31/517; A61K 31/455; A61K 31/506

(52) U.S. Cl. CPC A61K 31/7068; A61K 31/455; A61K 31/4985; A61K 31/4439; A61K 31/506; A61K 31/44; A61K 31/404; A61K 31/517; A61K 31/337
USPC 514/49; 514/355; 514/250; 514/340; 514/275; 514/266.4; 206/531

(57) ABSTRACT

In alternative embodiments the invention provides compositions, e.g., pharmaceutical compositions and preparations, formulations, kits and other products of manufacture, e.g., exemplary drug combinations packaged together or separately in blister packs, lidded blisters or blister cards, or wrapped in paper, plastic or cellophane wrappers (e.g., a shrink wrap), comprising combinations of beneficial ingredients that in alternative embodiments are serviceable as therapies or palliatives for treating, preventing or improving conditions, states and disease symptoms involving the use of targeted protein kinase inhibitors such as Multi-Kinase Inhibitors (MKIs) or Epidermal Growth Factor Receptor (EGFR) Inhibitors (EGFRIs), or the use of other oral oncolytics, such as capcitabine or erlotinib, known to be associated with drug-induced dermatological toxicity, e.g., in the amelioration or treatment of a cancer, a dermatitis, a rosacea, an eczema, an ichthyosis, or a related condition; and methods for making and using these compositions; and methods for making and using these compositions.

### FIELD OF THE INVENTION

This invention relates generally to medicine and pharmaceutical formulations. In alternative embodiments the invention provides compositions, e.g., pharmaceutical compositions and preparations, formulations, kits and other products of manufacture, e.g., exemplary drug combinations packaged together or separately in blister packs, lidded blisters or blister cards, or wrapped in paper, plastic or cellophane wrappers (e.g., a shrink wrap), comprising combinations of beneficial ingredients that in alternative embodiments are serviceable as therapies or palliatives for treating, preventing or improving conditions, states and disease symptoms involving the use of targeted protein kinase inhibitors such as Multi-Kinase Inhibitors (MKIs) or Epidermal Growth Factor Receptor (EGFR) Inhibitors (EGFRIs), or the use of other oral oncolytics, such as capcitabine or erlotinib, known to be associated with drug-induced dermatological toxicity, e.g., in the amelioration or treatment of a cancer, a dermatitis, a rosacea, an eczema, an ichthyosis, or a related condition; and methods for making and using these compositions; and methods for making and using these compositions.

### BACKGROUND

Single agent therapies have been used for diseases such as cancer; but these therapies can result in unwanted side effects, including inflammation, excessive sympathoneural drive, cachexia, anorexia, and stress or anxiety related thereto. The success achieved with the attempted escalation of doses of single agents has been limited; and the obstacles to increasing the single agent doses include exceeding the therapeutic windows and undesirable side effects at higher doses.

### SUMMARY

In alternative embodiments, the invention provides products of manufacture comprising a pharmaceutical composition or a formulation, a blister package, a lidded blister or a blister card or packet, a clamshell, a tray or a shrink wrap, or a kit, comprising:
(a) (i) a pharmaceutical composition or formulation comprising a nicotinamide, niacinamide or nicotinic acid amide, or an equivalent thereof; and
(ii) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof,
wherein optionally the fatty acid; ceramide or acylceramide, sphingosine-fatty acid, or equivalent thereof; and cholesterol, a (3β)-cholest-5-en-3-ol, or equivalent thereof, are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2;
(b) the product of manufacture of (a), wherein the composition or formulation comprises an ampoule, a gel, a lotion, a cream, an emollient, a skin patch or adhesive, aerosol or a spray for topical application,

wherein optionally the ampoule, gel, lotion, cream, emollient, skin patch or adhesive, aerosol or spray is packaged and/or formulated as a single unit dosage, optionally as one (a single) dosage of a gel, lotion, cream or emollient packaged in its own (is contained in a single (one)) tube, ampoule or packette;
(c) the product of manufacture of (a) or (b), wherein:
   (i) the formulation has between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: nicotinamide, niacinamide or nicotinic acid amide, or an equivalent thereof;
   (ii) the formulation has between about 1.0% to 10%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
   (iii) the formulation has between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids, wherein optionally the fatty acids comprise: at least two essential fatty acids, or alpha-linolenate and/or linoleate; a lecithin; an oleate, or an oleic acid, oleyl oleate or oleyl stearate; a palmitate, or a palmitate, palmitamine or palmitamide); a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate);
   (iv) the formulation has between about 0.1% to 10% (or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% or more) ceramide or acylceramide, wherein optionally the ceramide or acylceramide comprises a ceramide 1-9, or a ceramide or acylceramide derivative;
(d) the product of manufacture of any of (a) to (c), further comprising instructions for use;
(e) the product of manufacture of any of (a) to (d), further comprising: a urea (optionally as a urea cream) or a keratolytic; petrolatum or an occlusive; glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase; a formulation of white soft paraffin, a cetomacrogol and a cetostearyl alcohol; a tadalafil (optionally ADCIRCA™ or CIALIS™) (optionally to improve perfusion of distal vascular beds); a direct arterial vasodilator (optionally a minoxidil (optionally ROGAINE™)); or any combination thereof;
(f) the product of manufacture of any of (a) to (e), further comprising a pharmaceutical composition or formulation comprising a vasodilator,

wherein optionally the vasodilator comprises: an Angiotensin Converting Enzyme inhibitor (ACEi), an angiotensin receptor type II blocker, an embusartan, a fonsartan, a pratosartan, a phosphodiesterase subtype-selective inhibitor, a tadalafil (optionally ADCIRCA™ or CIALIS™), a vardenafil (optionally LEVITRA™), a direct vasodilator that blocks a K_{ATP} channel, a pinacidil, a naminidil, or a combination thereof,

wherein optionally:
the Angiotensin Converting Enzyme inhibitor (ACEi) is a captopril (optionally CAPOTEN™) (optionally formulated at 0.1% to 5.0%), an enalapril (optionally RENITEC™, VASOTEC™) (optionally formulated at 0.1% to 5.0%), a lisinopril (optionally PRINIVIL™, TENSOPRIL™, ZESTRIL™) (optionally formulated at 0.1% to 5.0%), or
the Angiotensin Converting Enzyme inhibitor (ACEi) is benazepril (optionally LOTENSIN™), captopril (optionally CAPOTEN™), cilazapril (optionally INHIBACE™, ZAPRIL™, VASCACE™), enalapril (optionally RENITEC™, VASOTEC™), enalaprilat, fosinopril (optionally MONOPRIL™), imidapril, lisinopril (optionally PRINIVIL™, TENSOPRIL™, ZESTRIL™), moexipril (optionally UNIVASC™, PERDIXS™), perindopril (optionally COVERSYL™, ACEON™), quinapril (optionally ACCUPRIL™), ramipril (optionally ALTACE™, PRILACE™) trandolapril (optionally MAVIK™) or a combination thereof,
the angiotensin receptor type II blockers is a losartan (optionally COZAAR™), optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; an embusartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; a fonsartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; or a pratosartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
the phosphodiesterase subtype-selective inhibitor is a sildenafil, optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; a tadalafil, optionally ADCIRCA™ or CIALIS™, optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; or a vardenafil (optionally LEVITRA™), optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
the direct vasodilator that blocks K_{ATP} channels is a minoxidil (optionally ROGAINE™) (optionally formulated at about 0.1% to 10%),
the pinacidil is optionally formulated at about 0.1% to 10%,
the naminidil is optionally formulated at about 0.1% to 10%;
(g) the product of manufacture of any of (a) to (f), further comprising an oral oncolytic, a targeted kinase inhibitor, a Multi Kinase Inhibitor (MKI), an Epidermal Growth Factor Receptor Inhibitor (EGFRI), a cancer drug, or a combination thereof,

wherein optionally the MKI is sorafenib (or NEXAVAR™), and optionally the MKI is formulated for a dose-escalation protocol, optionally comprising a 100 mg dose (⅛^{th} the approved dose) on days 1 to 3, 200 mg (¼^{th} the approved dose) on days 4 to 6, 200 mg twice per day (½ the approved dose) on days 7 to 9, and 400 mg twice per day on day 10 and beyond,

and optionally the MKI is a sunitinib (or SUTENT™),

and optionally the oral oncolytic is a capcitabine (XELODA™), optionally used at a dosage of about 150 mg, 300 mg, 500 mg, 1000 mg, 1500 mg, 2000 mg, 2500 mg, 3000 mg, 3500 mg, 4000 mg, 4500 mg, or 5000 mg,

and optionally the EGFRI is an erlotinib (or a TARCEVA™), optionally used at a dosage of about 25, 50, 75, 100, 125, 150, 175, 200, 225, or 250 mg, or between about 20 to 300 mg,

and optionally the cancer drug is a gemcitabine (or a GEMZAR™) or a docetaxel (or a TAXOTERE™);
(h) the product of manufacture of any of (a) to (g), further comprising an angiotensin converting enzyme inhibitor (ACEi), or an angiotensin receptor type II blocker;

wherein optionally the angiotensin converting enzyme inhibitor (ACEi) comprises a captopril (optionally CAPOTEN™) (optionally formulated at about 6.25 mg to 50 mg), an enalapril (optionally RENITEC™, VASOTEC™) (optionally formulated at about 1 mg to 100 mg), a lisinopril (optionally PRINIVIL™, TENSOPRIL™, ZESTRIL™) (optionally formulated at about 2.5 mg to 160 mg),

and optionally the angiotensin receptor type II blocker comprises a losartan (optionally COZAAR™) (optionally formulated at about 6.25 mg to 200 mg), an embusartan (optionally formulated for administration at about 0.1 to 30 mg/kg), a fonsartan (optionally formulated for administration at about 0.1 to 30 mg/kg), pratosartan (optionally formulated for administration at about 10 to 320 mg/day).

In alternative embodiments, the invention provides products of manufacture comprising a pharmaceutical composition or a formulation, a blister package, a lidded blister or a blister card or packet, a clamshell, a tray or a shrink wrap, or a kit, comprising:
(a) (i) a pharmaceutical composition or formulation comprising a vasodilator; and
(ii) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof,
wherein optionally the fatty acid; ceramide or acylceramide, sphingosine-fatty acid, or equivalent thereof; and cholesterol, a (3β)-cholest-5-en-3-ol, or equivalent thereof, are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2;
(b) the product of manufacture of (a), wherein the composition or formulation comprises an ampoule, a gel, a lotion, a cream, an emollient, a skin patch or adhesive, aerosol or a spray for topical application,

wherein optionally the ampoule, gel, lotion, cream, emollient, skin patch or adhesive, aerosol or spray is packaged and/or formulated as a single unit dosage, for example, one (a single) dosage of a gel, lotion, cream or emollient is packaged in its own (is contained in a single (one)) tube, ampoule or packette
(c) the product of manufacture of (a) or (b), wherein:
   (i) the vasodilator comprises: an Angiotensin Converting Enzyme inhibitor (ACEi), an angiotensin receptor type II blocker, an embusartan, a fonsartan, a pratosartan, a phosphodiesterase subtype-selective inhibitor, a tadalafil (optionally ADCIRCA™ or CIALIS™), a vardenafil (optionally LEVITRA™) (optionally LEVITRA™), a direct vasodilator that blocks a K_{ATP} channel, a pinacidil, a naminidil, or a combination thereof;
   wherein optionally:
   the Angiotensin Converting Enzyme inhibitor (ACEi) is a captopril (optionally CAPOTEN™) (optionally formulated at 0.1% to 5.0%), an enalapril (optionally RENITEC™, VASOTEC™) (optionally formulated at 0.1% to 5.0%), a lisinopril (optionally PRINIVIL™, TENSOPRIL™, ZESTRIL™) (optionally formulated at 0.1% to 5.0%), or
   the Angiotensin Converting Enzyme inhibitor (ACEi) is benazepril (optionally LOTENSIN™), captopril (optionally CAPOTEN™), cilazapril (optionally INHIBACE™, ZAPRIL™, VASCACE™), enalapril (optionally RENITEC™, VASOTEC™), enalaprilat, fosinopril (optionally MONOPRIL™), imidapril, lisinopril (optionally PRINIVIL™, TENSOPRIL™, ZESTRIL™), moexipril (optionally UNIVASC™, PERDIXS™), perindopril (optionally COVERSYL™, ACEON™), quinapril (optionally ACCUPRIL™), ramipril (optionally ALTACE™, PRILACE™) trandolapril (optionally MAVIK™) or a combination thereof;
   the angiotensin receptor type II blockers is a losartan (optionally COZAAR™), optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; an embusartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; a fonsartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; or a pratosartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
   the phosphodiesterase subtype-selective inhibitor is a sildenafil, optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; a tadalafil, optionally ADCIRCA™ or CIALIS™, optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; or a vardenafil (optionally LEVITRA™), optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more;
   the direct vasodilator that blocks K_{ATP} channels is a minoxidil (optionally ROGAINE™) (optionally formulated at about 0.1% to 10%);
   the pinacidil is optionally formulated at about 0.1% to 10%;
   the naminidil is optionally formulated at about 0.1% to 10%;
   (ii) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
   (iii) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids, wherein optionally the fatty acids comprise: at least two essential fatty acids, or alpha-linolenate and/or linoleate; a lecithin; an oleate, or an oleic acid, oleyl oleate or oleyl stearate; a palmitate, or a palmitate, palmitamine or palmitamide); a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate)
   (iv) the formulation has about 0.1% to 10% ceramide or acylceramide, wherein optionally the ceramide or acylceramide comprises a ceramide 1-9, or a ceramide derivative;
(d) the product of manufacture of any of (a) to (c), further comprising instructions for use; or
(e) the product of manufacture of any of (a) to (d), further comprising: a urea (optionally as a urea cream) or a keratolytic; a petrolatum or an occlusive; glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase; a formulation of white soft paraffin, a cetomacrogol and a cetostearyl alcohol; a tadalafil (optionally CIALIS™) (optionally to improve perfusion of distal vascular beds); a direct arterial vasodilator (optionally a minoxidil (optionally ROGAINE™)); or any combination thereof.

In alternative embodiments, the invention provides products of manufacture comprising a pharmaceutical composition or a formulation, a blister package, a lidded blister or a blister card or packet, a clamshell, a tray or a shrink wrap, or a kit, comprising:
(a) (i) a pharmaceutical composition or formulation comprising an angiotensin converting enzyme inhibitor (ACEi), or an angiotensin receptor type II blocker; and
(ii) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof,
wherein optionally the fatty acid; ceramide or acylceramide, sphingosine-fatty acid, or equivalent thereof; and cholesterol, a (3β)-cholest-5-en-3-ol, or equivalent thereof, are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2;
(b) the product of manufacture of (a), wherein the composition or formulation comprises an ampoule, a gel, a lotion, a cream, an emollient, a skin patch or adhesive, aerosol or a spray for topical application,

wherein optionally the ampoule, gel, lotion, cream, emollient, skin patch or adhesive, aerosol or spray is packaged and/or formulated as a single unit dosage, for example, one (a single) dosage of a gel, lotion, cream or emollient is packaged in its own (is contained in a single (one)) tube, ampoule or packette;
(c) the product of manufacture of (a) or (b), wherein:
   (i) the angiotensin converting enzyme inhibitor (ACEi) comprises a captopril (optionally CAPOTEN™) (optionally formulated at about 6.25 mg to 50 mg), an enalapril (optionally RENITEC™, VASOTEC™) (optionally formulated at about 1 mg to 100 mg), a lisinopril (optionally PRINIVIL™, TENSOPRIL™, ZESTRIL™) (optionally formulated at about 2.5 mg to 160 mg); or
   the angiotensin receptor type II blocker comprises a losartan (optionally COZAAR™) (optionally formulated at about 6.25 mg to 200 mg), an embusartan (optionally formulated for administration at about 0.1 to 30 mg/kg), a fonsartan (optionally formulated for administration at about 0.1 to 30 mg/kg), pratosartan (optionally formulated for administration at about 10 to 320 mg/day);
   (ii) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
   (iii) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids, wherein optionally the fatty acids comprise: at least two essential fatty acids, or alpha-linolenate and/or linoleate; a lecithin; an oleate, or an oleic acid, oleyl oleate or oleyl stearate; a palmitate, or a palmitate, palmitamine or palmitamide); a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate)
   (iv) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: ceramide or acylceramide, wherein optionally the ceramide or acylceramide comprises a ceramide 1-9, or a ceramide or acylceramide derivative;
(d) the product of manufacture of any of (a) to (c), further comprising instructions for use; or
(e) the product of manufacture of any of (a) to (d), further comprising: a urea (optionally as a urea cream) or a keratolytic; a petrolatum or an occlusive; glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase; a formulation of white soft paraffin, a cetomacrogol and a cetostearyl alcohol; a tadalafil (optionally CIALIS™) (optionally to improve perfusion of distal vascular beds); a direct arterial vasodilator (optionally a minoxidil (optionally ROGAINE™)); or any combination thereof.

In alternative embodiments, the invention provides products of manufacture comprising a pharmaceutical composition or a formulation, a blister package, a lidded blister or a blister card or packet, a clamshell, a tray or a shrink wrap, or a kit, comprising:
(a) (i) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof; or
(ii) the product of manufacture of (a)(i), wherein the fatty acid; ceramide or acylceramide, sphingosine-fatty acid, or equivalent thereof; and cholesterol, a (3β)-cholest-5-en-3-ol, or equivalent thereof, are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2;
(b) the product of manufacture of (a), wherein the composition or formulation comprises an ampoule, a gel, a lotion, a cream, an emollient, a skin patch or adhesive, aerosol or a spray for topical application,

wherein optionally the ampoule, gel, lotion, cream, emollient, skin patch or adhesive, aerosol or spray is packaged and/or formulated as a single unit dosage, for example, one (a single) dosage of a gel, lotion, cream or emollient is packaged in its own (is contained in a single (one)) tube, ampoule or packette;
(c) the product of manufacture of (a) or (b), wherein:
   (i) the formulation has between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: nicotinamide, niacinamide or nicotinic acid amide, or an equivalent thereof;
   (ii) the formulation has between about 1.0% to 10%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
   (iii) the formulation has between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids, wherein optionally the fatty acids comprise: at least two essential fatty acids, or alpha-linolenate and/or linoleate; a lecithin; an oleate, or an oleic acid, oleyl oleate or oleyl stearate; a palmitate, or a palmitate, palmitamine or palmitamide); a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate);
   (iv) the formulation has between about 0.1% to 10% (or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% or more) ceramide or acylceramide, wherein optionally the ceramide or acylceramide comprises a ceramide 1-9, or a ceramide or acylceramide derivative;
(d) the product of manufacture of any of (a) to (c), further comprising instructions for use;
(e) the product of manufacture of any of (a) to (d), further comprising: a urea (optionally as a urea cream) or a keratolytic; a petrolatum or an occlusive; glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase; a formulation of white soft paraffin, a cetomacrogol and a cetostearyl alcohol; a tadalafil (optionally CIALIS™) (optionally to improve perfusion of distal vascular beds); a direct arterial vasodilator (optionally a minoxidil (optionally ROGAINE™)); or any combination thereof;
(f) the product of manufacture of any of (a) to (e), further comprising an oral oncolytic, a targeted kinase inhibitor, a Multi Kinase Inhibitor (MKI), an Epidermal Growth Factor Receptor Inhibitor (EGFRI), a cancer drug, or a combination thereof,

wherein optionally the MKI is sorafenib (or NEXAVAR™), and optionally the MKI is formulated for a dose-escalation protocol, optionally comprising a 100 mg dose (⅛^{th} the approved dose) on days 1 to 3, 200 mg (¼^{th} the approved dose) on days 4 to 6, 200 mg twice per day (½ the approved dose) on days 7 to 9, and 400 mg twice per day on day 10 and beyond,

and optionally the MKI is a sunitinib (or SUTENT™),

and optionally the oral oncolytic is a capcitabine (XELODA™), optionally used at a dosage of about 150 mg, 300 mg, 500 mg, 1000 mg, 1500 mg, 2000 mg, 2500 mg, 3000 mg, 3500 mg, 4000 mg, 4500 mg, or 5000 mg,

and optionally the EGFRI is an erlotinib (or a TARCEVA™), optionally used at a dosage of about 25, 50, 75, 100, 125, 150, 175, 200, 225, or 250 mg, or between about 20 to 300 mg,

and optionally the cancer drug is a gemcitabine (or a GEMZAR™) or a docetaxel (or a TAXOTERE™);
(g) the product of manufacture of any of (a) to (f), further comprising an angiotensin converting enzyme inhibitor (ACEi), or an angiotensin receptor type II blocker;

wherein optionally the angiotensin converting enzyme inhibitor (ACEi) comprises a captopril (optionally CAPOTEN™) (optionally formulated at about 6.25 mg to 50 mg), an enalapril (optionally RENITEC™, VASOTEC™) (optionally formulated at about 1 mg to 100 mg), a lisinopril (optionally PRINIVIL™, TENSOPRIL™, ZESTRIL™) (optionally formulated at about 2.5 mg to 160 mg),

and optionally the angiotensin receptor type II blocker comprises a losartan (optionally COZAAR™) (optionally formulated at about 6.25 mg to 200 mg), an embusartan (optionally formulated for administration at about 0.1 to 30 mg/kg), a fonsartan (optionally formulated for administration at about 0.1 to 30 mg/kg), pratosartan (optionally formulated for administration at about 10 to 320 mg/day); or
(h) the product of manufacture of any of (a) to (g), further comprising a nicotinamide, niacinamide or nicotinic acid amide, or an equivalent thereof,
wherein optionally the nicotinamide, niacinamide or nicotinic acid amide, or an equivalent is formulated at about 4%, 5%, 6%, 7%, 8% or more, or about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: nicotinamide, niacinamide or nicotinic acid amide, or equivalent thereof; or
(i) the product of manufacture of any of (a) to (h), further comprising a pharmaceutical composition or formulation comprising a vasodilator,

wherein optionally the vasodilator comprises: an Angiotensin Converting Enzyme inhibitor (ACEi), an angiotensin receptor type II blocker, an embusartan, a fonsartan, a pratosartan, a phosphodiesterase subtype-selective inhibitor, a tadalafil (optionally CIALIS™), a vardenafil (optionally LEVITRA™), a direct vasodilator that blocks a K_{ATP} channel, a pinacidil, a naminidil, or a combination thereof,

wherein optionally:
the Angiotensin Converting Enzyme inhibitor (ACEi) is a captopril (optionally CAPOTEN™) (optionally formulated at 0.1% to 5.0%), an enalapril (optionally RENITEC™, VASOTEC™) (optionally formulated at 0.1% to 5.0%), a lisinopril (optionally PRINIVIL™, TENSOPRIL™, ZESTRIL™) (optionally formulated at 0.1% to 5.0%), or
the Angiotensin Converting Enzyme inhibitor (ACEi) is benazepril (optionally LOTENSIN™), captopril (optionally CAPOTEN™), cilazapril (optionally INHIBACE™, ZAPRIL™, VASCACE™), enalapril (optionally RENITEC™, VASOTEC™), enalaprilat, fosinopril (optionally MONOPRIL™), imidapril, lisinopril (optionally PRINIVIL™, TENSOPRIL™, ZESTRIL™), moexipril (optionally UNIVASC™, PERDIXS™), perindopril (optionally COVERSYL™, ACEON™), quinapril (optionally ACCUPRIL™), ramipril (optionally ALTACE™, PRILACE™) trandolapril (optionally MAVIK™) or a combination thereof,
the angiotensin receptor type II blockers is a losartan (optionally COZAAR™), optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; an embusartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; a fonsartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; or a pratosartan, optionally formulated at between about 0.1% to 5.0%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
the phosphodiesterase subtype-selective inhibitor is a sildenafil, optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; a tadalafil, optionally ADCIRCA™ or CIALIS™, optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; or a vardenafil (optionally LEVITRA™), optionally formulated at between about 0.1% to 10%, or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
the direct vasodilator that blocks K_{ATP} channels is a minoxidil (optionally ROGAINE™) (optionally formulated at about 0.1% to 10%),
the pinacidil is optionally formulated at about 0.1% to 10%,
the naminidil is optionally formulated at about 0.1% to 10%.

In alternative embodiments, the invention provides methods for administering a Multi Kinase Inhibitor (MKI) to an individual in need thereof to minimize the toxicity of the MKI to skin comprising:

(a) administering the Multi Kinase Inhibitor (MKI) in a dose-escalation regimen in step-wise in doses (dosages),

wherein the initial dose is a fraction of the MKI's approved dose, and optionally the initial dose is about 1/10^{th}, 1/9^{th}, ⅛^{th}, 1/7^{th}, ⅙^{th}, ⅕^{th}, ¼^{th}, ⅓^{th} or ½^{th} the approved dose,

and timing and duration of subsequent dosages is:
(i) determined as a function of the pharmacokinetic elimination properties of the individual MKI such that sufficient time is allowed for the MKI to approach a steady-state level in blood prior to advancing to a next or a final step,
(ii) titrated such that serum concentration of the MKI is increased to approach a steady-state (serum) level over a period determined by the pharmacokinetic elimination (clearance) of the administered MKI, or
(iii) a dose-escalation period sufficient to allow the skin to adapt to a new steady-state concentration of the MKI,

wherein (i), (ii) and (iii) are designed to allow the skin to become tolerant to the toxic effects of the MKIs, thereby improving the therapeutic ratio of the MKI;

(b) the method of (a) wherein the individual in need thereof is a human, and optionally a human having MKI cancer therapy, or the individual in need thereof has a dermatitis (e.g., a contact, an atopic, a seborrhoeic, a stasis, a perioral or other dermatitis), a rosacea (e.g., an erythematotelangiectatic, papulopustular, phymatous or ocular rosacea), an eczema (e.g., an atopic, contact, xerotic or seborrhoeic eczema), an ichthyosis (e.g., an epidermolytic hyperkeratosis or a lamellar ichthyosis), an actinic dermatitis (e.g., photosensitive eczema or chronic photosensitivity dermatitis), a hand dermatitis (e.g., a dyshidrosis, or acute vesiculobullous hand eczema), or a related condition;

(c) the method of (a) or (b), wherein the MKI is sorafenib (or NEXAVAR™), and optionally the dose-escalation protocol comprises a 100 mg dose (⅛^{th} the approved dose) on days 1 to 3, 200 mg (¼^{th} the approved dose) on days 4 to 6, 200 mg twice per day (½ the approved dose) on days 7 to 9, and 400 mg twice per day on day 10 and beyond;

(d) the method of (a) or (b), wherein the MKI comprises a regorafenib (optionally BAY 734506™, Bayer AG, Leverkusen, Germany), and optionally the dose-escalation protocol comprises a 20 mg dose on days 1 to 5, a 40 mg dose on days 6 to 10, an 80 mg dose on days 11 to 15, a 160 mg dose on day 16 to 20, and a 320 mg dose on day 21 and beyond—optionally, for the duration of therapy; or

(e) the method of (a) or (b), wherein the MKI is a sunitinib (or SUTENT™).

In alternative embodiments, the invention provides products of manufacture comprising a pharmaceutical composition or a formulation, a blister package, a lidded blister or a blister card or packet, a clamshell, a tray or a shrink wrap, or a kit, comprising all ingredients to practice a method of the invention and optionally further comprising instructions for use, wherein optionally the instructions comprise instructions for practicing all or part of a method of the invention, e.g., methods for administering an oral oncolytic, a targeted kinase inhibitor, a Multi Kinase Inhibitor (MKI), an Epidermal Growth Factor Receptor Inhibitor (EGFRI), a cancer drug, or a combination thereof to an individual in need thereof to minimize the toxicity of the comprising an oral oncolytic, a targeted kinase inhibitor, a Multi Kinase Inhibitor (MKI), an Epidermal Growth Factor Receptor Inhibitor (EGFRI), a cancer drug, or a combination thereof, to skin. In alternative embodiments, the individual in need thereof has a dermatitis (e.g., a contact, an atopic, a seborrhoeic, a stasis, a perioral or other dermatitis), a rosacea (e.g., an erythematotelangiectatic, papulopustular, phymatous or ocular rosacea), an eczema (e.g., an atopic, contact, xerotic or seborrhoeic eczema), an ichthyosis (e.g., an epidermolytic hyperkeratosis or a lamellar ichthyosis), an actinic dermatitis (e.g., photosensitive eczema or chronic photosensitivity dermatitis), a hand dermatitis (e.g., a dyshidrosis, or acute vesiculobullous hand eczema), or a related condition.

In alternative embodiments, the invention provides methods for improving hemoperfusion in tissues such as distal vascular beds, e.g., such as those found in skin, the palms and soles; mitigating Multi Kinase Inhibitor (MKI) toxicity in the skin, and/or for inhibiting local renin-angiotensin-aldosterone signaling that may contribute to MKI toxicity in the skin, comprising

(a) administering to an individual in need thereof:
(i) a pharmaceutical composition or formulation comprising an angiotensin converting enzyme inhibitor (ACEi), or an angiotensin receptor type II blocker; and
(ii)
(A) a composition or formulation comprising a physiologically balanced lipid formulation, wherein the composition or formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof,
wherein optionally the fatty acid; ceramide or acylceramide, sphingosine-fatty acid, or equivalent thereof; and cholesterol, a (3β)-cholest-5-en-3-ol, or equivalent thereof, are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2; or
(B) the Multi Kinase Inhibitor (MKI) in a dose-escalation regimen in step-wise in doses (dosages); or
(C) a combination of (A) and (B);

(b) the method of (a), wherein the composition or formulation comprises an ampoule, a gel, a lotion, a cream, an emollient, a skin patch or adhesive, aerosol or a spray for topical application,

wherein optionally the ampoule, gel, lotion, cream, emollient, skin patch or adhesive, aerosol or spray is packaged and/or formulated as a single unit dosage, for example, one (a single) dosage of a gel, lotion, cream or emollient is packaged in its own (is contained in a single (one)) tube, ampoule or packette;

(c) the method of (a) or (b), wherein:
(i) the angiotensin converting enzyme inhibitor (ACEi) comprises a captopril (optionally CAPOTEN™) (optionally formulated at about 6.25 mg to 50 mg), an enalapril (optionally RENITEC™, VASOTEC™) (optionally formulated at about 1 mg to 100 mg), a lisinopril (optionally PRINIVIL™, TENSOPRIL™, ZESTRIL™) (optionally formulated at about 2.5 mg to 160 mg); or
the angiotensin receptor type II blocker comprises a losartan (optionally COZAAR™) (optionally formulated at about 6.25 mg to 200 mg), an embusartan (optionally formulated for administration at about 0.1 to 30 mg/kg), a fonsartan (optionally formulated for administration at about 0.1 to 30 mg/kg), pratosartan (optionally formulated for administration at about 10 to 320 mg/day);
(ii) the formulation has about between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
(iii) the formulation has about between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids, wherein optionally the fatty acids comprise: at least two essential fatty acids, or alpha-linolenate and/or linoleate; a lecithin; an oleate, or an oleic acid, oleyl oleate or oleyl stearate; a palmitate, or a palmitate, palmitamine or palmitamide; a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate;
(iv) the formulation has about between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: ceramide or acylceramide, wherein optionally the ceramide or acylceramide comprises a ceramide 1-9, or a ceramide or acylceramide derivative;

(d) the method of any of (a) to (c), further comprising instructions for use; or

(e) the method of any of (a) to (d), wherein the composition or formulation further comprises: a urea (optionally as a urea cream) or a urea (optionally as a urea cream) or a keratolytic; a petrolatum or an occlusive; glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase; a formulation of white soft paraffin, a cetomacrogol and a cetostearyl alcohol; a tadalafil (optionally CIALIS™) (optionally to improve perfusion of distal vascular beds); a direct arterial vasodilator (optionally a minoxidil (optionally ROGAINE™)); or any combination thereof.

In alternative embodiments, the invention provides products of manufacture comprising a pharmaceutical composition or a formulation, a blister package, a lidded blister or a blister card or packet, a clamshell, a tray or a shrink wrap, or a kit, comprising all ingredients to practice a method of the invention; and optionally further comprising instructions for use, wherein optionally the instructions comprise instructions for practicing all or part of the method of the invention, e.g., methods for improving hemoperfusion in tissues such as distal vascular beds, e.g., such as those found in skin, the palms and soles; mitigating Multi Kinase Inhibitor (MKI) toxicity in the skin, and/or for inhibiting local renin-angiotensin-aldosterone signaling that may contribute to MKI toxicity in the skin.

In alternative embodiments, the invention provides methods for administering an oral oncolytic, a targeted kinase inhibitor, a Multi Kinase Inhibitor (MKI), an Epidermal Growth Factor Receptor Inhibitor (EGFRI), a cancer drug, or a combination thereof, to an individual in need thereof to minimize the toxicity of the oral oncolytic, a targeted kinase inhibitor, a Multi Kinase Inhibitor (MKI), an Epidermal Growth Factor Receptor Inhibitor (EGFRI), a cancer drug, or a combination thereof, to skin comprising:

(a) administering the oral oncolytic, a targeted kinase inhibitor, a Multi Kinase Inhibitor (MKI), an Epidermal Growth Factor Receptor Inhibitor (EGFRI), a cancer drug, or a combination thereof, optionally in a dose-escalation regimen in step-wise in doses (dosages),

wherein the initial dose is a fraction of the MKI's approved dose, and optionally the initial dose is about 1/10th, 1/9th, ⅛th, 1/7th, ⅙th, ⅕th, ¼th, ⅓th or ½th the approved dose; and

(b) administering a pharmaceutical composition or a formulation comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof.

In alternative embodiments, the invention provides methods for administering an oral oncolytic, a targeted kinase inhibitor, a Multi Kinase Inhibitor (MKI), an Epidermal Growth Factor Receptor Inhibitor (EGFRI), a cancer drug, or a combination thereof, to an individual in need thereof to minimize the toxicity of the oral oncolytic, a targeted kinase inhibitor, a Multi Kinase Inhibitor (MKI), an Epidermal Growth Factor Receptor Inhibitor (EGFRI), a cancer drug, or a combination thereof, to skin comprising:

(i) (a) administering a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof;

(b) administering a pharmaceutical composition or formulation comprising a angiotensin converting enzyme inhibitor (ACEi) captopril (optionally CAPOTEN™), optionally at a dose of 25 mg t. i. d or b. i. d, or at a single unit dosage of between about 20 to 60 mg t.i.d or b.i.d; and

(c) administering a an oral oncolytic, a targeted kinase inhibitor, a Multi Kinase Inhibitor (MKI), an Epidermal Growth Factor Receptor Inhibitor (EGFRI), a cancer drug, or a combination thereof, in a dose-escalation regimen in step-wise in doses (dosages), wherein the initial dose is a fraction of the approved dose, and optionally the initial dose is about 1/10th, 1/9th, ⅛th, 1/7th, ⅙th, ⅕th, ¼th, ⅓th or ½th the approved dose; or

(ii) the method of (i), further comprising administering a pharmaceutical composition or formulation comprising a nicotinamide, niacinamide or nicotinic acid amide, or an equivalent thereof.

In alternative embodiments, the invention provides products of manufacture comprising a pharmaceutical composition or a formulation, a blister package, a lidded blister or a blister card or packet, a clamshell, a tray or a shrink wrap, or a kit, comprising:

(a) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof; and

(b) a pharmaceutical composition or formulation comprising a nicotinamide, niacinamide or nicotinic acid amide, or an equivalent thereof.

In alternative embodiments, the invention provides products of manufacture comprising a pharmaceutical composition or a formulation, a blister package, a lidded blister or a blister card or packet, a clamshell, a tray or a shrink wrap, or a kit, comprising:

(i) (a) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof, and,

(b) a pharmaceutical composition or formulation comprising a nicotinamide, niacinamide or nicotinic acid amide, or an equivalent thereof; or

(ii) the product of manufacture of (a), further comprising a urea (optionally as a urea cream) and/or a keratolytica urea; a petrolatum or an occlusive; a glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase, or any combination thereof.

In alternative embodiments, the invention provides products of manufacture comprising a pharmaceutical composition or a formulation, a blister package, a lidded blister or a blister card or packet, a clamshell, a tray or a shrink wrap, or a kit, comprising:

(i) (a) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof, and,

(b) a pharmaceutical composition or formulation comprising a nicotinamide, niacinamide or nicotinic acid amide, or an equivalent thereof; or

(ii) the product of manufacture of (a), further comprising a tadalafil (optionally CIALIS™) or equivalent, or a drug to improve perfusion of distal vascular beds.

In alternative embodiments, the invention provides products of manufacture comprising a pharmaceutical composition or a formulation, a blister package, a lidded blister or a blister card or packet, a clamshell, a tray or a shrink wrap, or a kit, comprising:

(i) (a) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof, and,

(b) a pharmaceutical composition or formulation comprising a nicotinamide, niacinamide or nicotinic acid amide, or an equivalent thereof; or,

(ii) the product of manufacture of (a), further comprising a direct arterial vasodilator (optionally a minoxidil (optionally ROGAINE™)).

In alternative embodiments, the invention provides methods for administering an oral oncolytic, a targeted kinase inhibitor, a Multi Kinase Inhibitor (MKI), an Epidermal Growth Factor Receptor Inhibitor (EGFRI), a cancer drug, or a combination thereof, to an individual in need thereof to minimize the toxicity of the oral oncolytic, a targeted kinase inhibitor, a Multi Kinase Inhibitor (MKI), an Epidermal Growth Factor Receptor Inhibitor (EGFRI), a cancer drug, or a combination thereof, to skin comprising:

(i) (a) administering a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof;

(b) administering a pharmaceutical composition or formulation comprising a nicotinamide, niacinamide or nicotinic acid amide, or an equivalent thereof; and,

(c) administering an oral oncolytic, a targeted kinase inhibitor, a Multi Kinase Inhibitor (MKI), an Epidermal Growth Factor Receptor Inhibitor (EGFRI), a cancer drug, or a combination thereof, in a dose-escalation regimen in step-wise in doses (dosages), wherein the initial dose is a fraction of the approved dose, and optionally the initial dose is about 1/10th, 1/9th, ⅛th, 1/7th, ⅙th, ⅕th, ¼th, ⅓th or ½th the approved dose; or

(ii) the method of (i), further comprising administering:

(1) a urea (optionally as a urea cream) and/or a keratolytic; a petrolatum or an occlusive; a glycerol or a humectant; a citric acid or a pH buffer; one or more tocopherol(s) or an anti-oxidant; a retinoid (optionally a tazarotene); a glucocorticoid (optionally clobetasol propionate); a vasodilator; a diprobase, or a combination thereof;

(2) a direct arterial vasodilator (optionally a minoxidil (optionally ROGAINE™));

(3) a tadalafil (optionally CIALIS™) or equivalent, or a drug to improve perfusion of distal vascular beds; or

(d) any combination or all of (1) to (3) (optionally urea and minoxidil (optionally ROGAINE™)), or urea and tadalafil (optionally CIALIS™), or minoxidil and tadalafil, or urea, minoxidil and tadalafil).

In alternative embodiments, the invention provides methods for administering an oral oncolytic, a targeted kinase inhibitor, a Multi Kinase Inhibitor (MKI), an Epidermal Growth Factor Receptor Inhibitor (EGFRI), a cancer drug, or a combination thereof, to an individual in need thereof to minimize the toxicity of the MKI to skin comprising:

(i) (a) administering a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof;

(b) administering a pharmaceutical composition or formulation comprising a nicotinamide, niacinamide or nicotinic acid amide, or an equivalent thereof;

(c) administering an oral oncolytic, a targeted kinase inhibitor, a Multi Kinase Inhibitor (MKI), an Epidermal Growth Factor Receptor Inhibitor (EGFRI), a cancer drug, or a combination thereof in a dose-escalation regimen in step-wise in doses (dosages), wherein the initial dose is a fraction of the approved dose, and optionally the initial dose is about 1/10th, 1/9th, ⅛th, 1/7th, ⅙th, ⅕th, ¼th, ⅓th or ½th the approved dose; and

(d) administering a pharmaceutical composition or formulation comprising a angiotensin converting enzyme inhibitor (ACEi) captopril (optionally CAPOTEN™), optionally at a dose of 25 mg t.i.d or b.i.d, or at a single unit dosage of between about 20 to 60 mg t.i.d or b.i.d.; or

(ii) the method of (i), further comprising administering:

(1) a urea (optionally as a urea cream) and/or a keratolytic, a petrolatum or an occlusive; a glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase, or a combination thereof;

(2) a direct arterial vasodilator (optionally a minoxidil (optionally ROGAINE™));

(3) a tadalafil (optionally CIALIS™) or equivalent, or a drug to improve perfusion of distal vascular beds; or

(d) any combination or all of (1) to (3) (optionally urea and minoxidil (optionally ROGAINE™), or urea and tadalafil (optionally CIALIS™), or minoxidil and tadalafil, or urea, minoxidil and tadalafil).

In alternative embodiments, the invention provides products of manufacture comprising a pharmaceutical composition or a formulation, a blister package, a lidded blister or a blister card or packet, a clamshell, a tray or a shrink wrap, or a kit, comprising:

(a) a petrolatum or an occlusive; a glycerol or a humectant; a citrate or a physiological proton buffer; a tocopherol or a physiological anti-oxidant; a urea or a keratolytic; a mixture of lipids in a physiologically-balanced 1:2:1 mixture that comprises a fatty acid, wherein optionally the fatty acid comprises a linoleic or linolenic acid, a ceramide, acylceramide or a mixture of ceramides or acylceramides (wherein optionally the ceramide or acylceramide comprises a ceramide 1-9, or a ceramide or acylceramide derivative), a cholesterol; and a nicotinamide or a form of a niacin, a pyridine-3-carboxylic acid, or a vitamin B3;

(b) the product of manufacture of (a) comprising: between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more petrolatum or an occlusive; between about 1.0% to 10%, or between about 1.0% to 20% glycerol or a humectant; between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more citrate or a physiological proton buffer; between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more tocopherol or a physiological anti-oxidant; between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more urea or a keratolytic; between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more lipids, optionally in a physiologically-balanced 1:2:1 mixture that includes about 1.25% fatty acids, optionally a linoleic or linolenic acid, 2.5% ceramide, acylceramide or a mixture of ceramides or acylceramides, 1.25% cholesterol; and between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more nicotinamide or a form of a niacin, a pyridine-3-carboxylic acid, or a vitamin B3; or

(c) the product of manufacture of (b) comprising: about 5% petrolatum or an occlusive; 20% glycerol or a humectant; 0.5% citrate or a physiological proton buffer; 1% tocopherol or a physiological anti-oxidant; 5% urea or a keratolytic; 5% lipids in a physiologically-balanced 1:2:1 mixture that includes 1.25% fatty acids, especially a linoleic or linolenic acid, 2.5% a ceramide, acylceramide or a mixture of ceramides or acylceramides, 1.25% cholesterol; and about 1% nicotinamide or a form of a niacin, a pyridine-3-carboxylic acid, or a vitamin B3.

The invention provides blister packs or a plurality of blister packettes comprising a therapeutic combination of or pharmaceutical composition of the invention, wherein the ingredients (e.g., drugs, lotions, gels, etc.) are arranged or clustered in the blister pack or a plurality of blister packettes: (a) in a chrono-dosing arrangement or pattern; or (b) individually.

In alternative embodiments, the invention provides paper, plastic, cellophane package, polyvinyl chloride (PVC) plastic and/or an aluminium foil (alufoil) material, or a plurality of packettes comprising a therapeutic combination of or pharmaceutical composition of the invention, wherein the ingredients (e.g., drugs, lotions, gels, etc.) are arranged or clustered in the package or a plurality of packettes: (a) in a chrono-dosing arrangement or pattern; or (b) individually.

In alternative embodiments, the invention provides methods for administering an oral oncolytic, a targeted kinase inhibitor, a Multi Kinase Inhibitor (MKI), an Epidermal Growth Factor Receptor Inhibitor (EGFRI), a cancer drug, or a combination thereof, to an individual in need thereof to minimize the toxicity of the oral oncolytic, a targeted kinase inhibitor, a Multi Kinase Inhibitor (MKI), an Epidermal Growth Factor Receptor Inhibitor (EGFRI), a cancer drug, or a combination thereof, to skin comprising administering a product of manufacture of the invention to an individual in need thereof, and optionally the individual in need thereof is being treated for a cancer, or the individual in need thereof has a dermatitis (e.g., a contact, an atopic, a seborrhoeic, a stasis, a perioral or other dermatitis), a rosacea (e.g., an erythematotelangiectatic, papulopustular, phymatous or ocular rosacea), an eczema (e.g., an atopic, contact, xerotic or seborrhoeic eczema), an ichthyosis (e.g., an epidermolytic hyperkeratosis or a lamellar ichthyosis), an actinic dermatitis (e.g., photosensitive eczema or chronic photosensitivity dermatitis), a hand dermatitis (e.g., a dyshidrosis, or acute vesiculobullous hand eczema), or a related condition.

The details of one or more aspects of the invention are set forth in the description below. Other features, objects, and advantages of the invention will be apparent from the description and from the claims.

All publications, patents and patent applications cited herein are hereby expressly incorporated by reference for all purposes.

### DETAILED DESCRIPTION

In alternative embodiments the invention provides compositions, including pharmaceutical compositions and preparations, formulations, kits and other products of manufacture, e.g., exemplary drug and formulation combinations packaged together or separately in products of manufacture, e.g., as blister packs or packettes, lidded blisters or blister cards, or wrapped in paper, aluminum, plastic or cellophane wrappers (e.g., a shrink wrap), comprising combinations of beneficial ingredients of the invention. In alternative embodiments, the combinations of beneficial ingredients of the invention allow for escalation of doses of single or combinations of agents (e.g., cancer drugs). In alternative embodiments, the combinations of beneficial ingredients of the invention overcomes obstacles to increasing single agent doses to exceed what would otherwise be “a therapeutic window”, thus mitigating (decreasing or eliminating) undesirable side effects at these higher doses (e.g., dosages that would not be administered (e.g., because of undesirable or unacceptable side effects) without also practicing a method of the invention or administering a composition of the invention). In alternative embodiments, the combinations of beneficial ingredients of the invention, and methods of the invention, are used as (or with) therapies or as palliatives for treating, preventing and/or improving conditions, states and disease symptoms involving use of oral oncolytics, targeted kinase inhibitors, Multi Kinase Inhibitors (MKI), Epidermal Growth Factor Receptor Inhibitors (EGFRI), a cancer drug, or a combination thereof, e.g., in the treatment or amelioration of a cancer, a dermatitis (e.g., a contact, an atopic, a seborrhoeic, a stasis, a perioral or other dermatitis), a rosacea (e.g., an erythematotelangiectatic, papulopustular, phymatous or ocular rosacea), an eczema (e.g., an atopic, contact, xerotic or seborrhoeic eczema), an ichthyosis (e.g., an epidermolytic hyperkeratosis or a lamellar ichthyosis), an actinic dermatitis (e.g., photosensitive eczema or chronic photosensitivity dermatitis), a hand dermatitis (e.g., a dyshidrosis, or acute vesiculobullous hand eczema), or a related condition; and methods for making and using these compositions.

Dose-Escalation or Continuous Dosing Regimens of the Invention

In alternative embodiments, the invention provides compositions and methods comprising a dose-escalation of, or continuous dosing of: an oral oncolytic, a targeted kinase inhibitor, a Multi Kinase Inhibitor (MKI), an Epidermal Growth Factor Receptor Inhibitor (EGFRI), a cancer drug, or a combination thereof, used, e.g., in a cancer therapy, or a therapy for a dermatitis (e.g., a contact, an atopic, a seborrhoeic, a stasis, a perioral or other dermatitis), a rosacea (e.g., an erythematotelangiectatic, papulopustular, phymatous or ocular rosacea), an eczema (e.g., an atopic, contact, xerotic or seborrhoeic eczema), an ichthyosis (e.g., an epidermolytic hyperkeratosis or a lamellar ichthyosis), an actinic dermatitis (e.g., photosensitive eczema or chronic photosensitivity dermatitis), a hand dermatitis (e.g., a dyshidrosis, or acute vesiculobullous hand eczema), or a related condition.

MKIs can block multiple signaling pathways important to maintaining a tissue proliferative unit and a vascular proliferative unit in skin; and in one embodiment the invention's compositions and methods minimize the toxicity of MKIs in tissues, e.g., in skin, by providing drug dose-escalation protocols and or continuous dosing protocols and compositions (e.g., kits) for use in both or either protocols. In one embodiment, the MKIs are administered step-wise in doses that begin at a fraction of the approved dose. In one embodiment, the timing and duration of the steps is determined as a function of the pharmacokinetic elimination properties of the individual MKI agent such that sufficient time is allowed for the drug to approach a steady-state level in the blood prior to advancing to the next or final step.

For example, one embodiment comprises a dose escalation of the MKI sorafenib, which has a pharmacokinetic half-life of 20 to 24 hrs; this exemplary dose-escalation protocol comprises a 100 mg dose (⅛^{th} the approved dose) on days 1 to 3, 200 mg (¼^{th} the approved dose) on days 4 to 6, 200 mg twice per day (½ the approved dose) on days 7 to 9, and 400 mg twice per day on day 10 and beyond. In another exemplary embodiment the MKI is sorafenib (or NEXAVAR™), and optionally the dose-escalation protocol comprises a 100 mg dose (⅛th the approved dose) on days 1 to 3, 200 mg (¼th the approved dose) on days 4 to 6, 200 mg twice per day (½ the approved dose) on days 7 to 9, 400 mg twice per day on days 10 to 13 (per the prescribing information), and 800 mg twice per day (twice the approved dose) on day 14 and beyond. In another exemplary embodiment the MKI is regorafenib, and optionally the dose-escalation protocol comprises a 20 mg dose on days 1 to 5, a 40 mg dose on days 6 to 10, an 80 mg dose on days 11 to 15, a 160 mg dose on day 16 to 20, and a 320 mg dose on day 21 and longer, and optionally, this lasting beyond for the duration of therapy.

Another exemplary alternative embodiment comprises a protocol for increasing the serum concentration of an MKI such that it approached the steady-state level over a period determined by the pharmacokinetic elimination of a given MKI. A third exemplary alternative would entail a dose-escalation period sufficient to allow the skin to adapt to a new steady-state concentration of the MKI.

Each alternative is designed to allow the skin to become tolerant to the toxic effects of the MKIs, thereby improving the therapeutic ratio of the MKI.

In alternative embodiments, the invention provide compositions and methods for use with an oral oncolytic, a targeted kinase inhibitor, a targeted Multi Kinase Inhibitor (MKI), an Epidermal Growth Factor Receptor Inhibitor (EGFRI), a cancer drug, or a combination thereof, which are emerging as important tools to reduce tumor progression and increase overall survival in cancer patients. While these drugs are generally well-tolerated in the context of oncolytic therapies, they often have dose-limiting dermatological toxicity; and in one embodiment, the compositions and methods of this invention allow for a dose escalation of e.g., MKIs, an oral oncolytic, a targeted kinase inhibitor, a targeted Multi Kinase Inhibitor (MKI), an Epidermal Growth Factor Receptor Inhibitor (EGFRI), a cancer drug, or a combination thereof. For example, compositions and methods of the invention are used to address (treat or ameliorate) a dermatological toxicity such as a Hand Foot Skin Reaction (HFSR) or papulo-pustular erythrodysesthesia (PPE), a significant class-specific dermatologic dose-limiting toxicity associated with certain oral oncolytics, MKIs and the like. In alternative embodiments, the invention provide compositions and methods enabling a dose escalation strategy with an adjunct treatment regimen to prevent or to decrease the severity of a dermatological toxicity or HFSR or papulo-pustular erythrodysesthesia (PPE).

While the invention is not limited by any particular mechanism of action, this therapeutic approach of compositions and methods of the invention is based on first principles of physiology and pharmacology applied to palmar-plantar tissue compartments. HFSR is a maladaptive wound healing response to acute and chronic tissue injury. The acute injury is caused by MKI levels that exceed the ability of palmar-plantar tissue to adapt to the blockade of growth factor signaling pathways involved in normal growth and differentiation. The chronic injury is caused by mechanical stress exceeding the impaired pressure barrier capacity of the palmar-plantar tissue. The maladaptive wound healing response is intrinsic to the therapeutic effect of MKI, and therefore prevention of tissue injury (as can be done by practicing compositions and methods of the invention) is required to avoid treatment disrupting dose adjustment and/or treatment interruption. As such, compositions and methods of the invention provide a novel dosing and adjunct treatment regimen to ameliorate and/or prevent acute and chronic tissue injury subsequent to treatment using an oral oncolytic, a targeted kinase inhibitor, a Multi Kinase Inhibitor (MKI), an Epidermal Growth Factor Receptor Inhibitor (EGFRI), a cancer drug, or a combination thereof, and enable patients to benefit from maximum cumulative (e.g., MKI, oral oncolytic, etc.) dosing without treatment interruption.

While the invention is not limited by any particular mechanism of action, the MKIs are associated with a dermatological toxicity or HFSR, a toxicity thought to be related to the mechanism of action of these drugs, and particularly the inhibition of VEGF and PDGF signaling. The compositions and methods of the invention provides acute and chronic prophylaxis for a dermatological toxicity or a HFSR. The compositions and methods of the invention provide a prophylaxis and prevention that may be preferable to reactive treatment once dermatological toxicity appears, especially because the impaired wound healing response is intrinsic to the therapeutic efficacy of MKIs.

In alternative embodiments compositions and methods of the invention provide:
a dose-escalation strategy to minimize acute MKI-induced trauma, or trauma due to administration of an oral oncolytic, a targeted kinase inhibitor, a Multi Kinase Inhibitor (MKI), an Epidermal Growth Factor Receptor Inhibitor (EGFRI), a cancer drug, or a combination thereof, and allow keratinocytes time to adapt to inhibition of target kinases without substantively reducing oncolytic efficacy; and
a combination product comprised of (comprising) a systemically administered inhibitor of the renin-angiotensin system to mitigate the acute injury response and improve barrier function with improve hemodynamics in skin and a topically administered physiological balanced emollient to improve pressure barrier function, reduce hyperkeratosis and metabolic load, and increase tolerance to mechanical stress.

Systemic Inhibitors of Renin-Angiotensin-Aldosterone Signaling

In alternative embodiments, the invention provides compositions and methods for administering systemic inhibitors of renin-angiotensin-aldosterone signaling. In alternative embodiments, inhibitors of renin-angiotensin-aldosterone signaling are given systemically to improve hemoperfusion of distal vascular beds such as those found in skin, especially the palms and soles; and optionally to inhibit local renin-angiotensin-aldosterone signaling that may contribute to MKI toxicity in the skin.

In alternative embodiments, compositions and methods of the invention comprise use (administration) of angiotensin converting enzyme inhibitors (ACEi) such as captopril (optionally CAPOTEN™) (optionally formulated for administration at 6.25 mg up to 50 mg three times per day), enalapril (optionally RENITEC™, VASOTEC™) (optionally formulated for administration at 1 mg up to 100 mg per day), lisinopril (optionally PRINIVIL™, TENSOPRIL™, ZESTRIL™) (optionally formulated for administration at 2.5 mg up to 160 mg per day); and in alternative embodiments, comprise use of: angiotensin receptor type II blockers such as losartan (optionally COZAAR™) (optionally formulated for administration at 6.25 mg up to 200 mg per day), embusartan (optionally formulated for administration at 0.1 to 30 mg/kg), fonsartan (optionally formulated for administration at 0.1 to 30 mg/kg), pratosartan (optionally formulated for administration at 10 to 320 mg/day).

In alternative embodiments, compositions and methods of the invention comprise use (administration) of angiotensin converting enzyme inhibitors (ACEi) in combination with: a dose-escalation protocol (method) of the invention; a topical gel, lotion or emollient, including the formulations, gels, lotions, emollients, skin patches or adhesives, aerosols and sprays of this invention; and, any combination thereof.

Topical Emollient Creams, Gels, Lotions, Skin Patches or Adhesives, Aerosols, Sprays

In alternative embodiments, the invention provides compositions and methods for administering topical emollient creams, lotions, gels, skin patches or adhesives, aerosols and sprays and the like, alone or in combination with other formulations or pharmaceutical compositions. In alternative embodiments, the stress imposed on the skin by the oral oncolytic, a targeted kinase inhibitor, a Multi Kinase Inhibitor (MKI), an Epidermal Growth Factor Receptor Inhibitor (EGFRI), a cancer drug, or a combination thereof, is ameliorated (e.g., partially alleviated) through the use of a topical formulation, e.g., an emollient cream, gel, lotions, skin patches or adhesives, aerosols or sprays and the like, designed to improve the barrier function of skin, reduce hyperkeratosis, and/or decrease metabolic load.

In alternative embodiments, the topical formulation, e.g., lotions, emollient cream, gel, skin patches or adhesives, aerosols or spray and the like, comprises the three main categories of lipids essential to maintaining skin barrier function:
a cholesterol (optionally formulated at 1.0% to 10% (or about 1%, 2%, 3%, 4%, 5%, 6%, 7%, 8%, 9%, 10% or more));
a fatty acid (optionally formulated at between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more), including e.g. the two essential fatty acids (alpha-linolenate and linoleate), lecithins, oleates (e.g., oleic acid, oleyl oleate, oleyl stearate), palmitates (e.g., palmitate, palmitamine, palmitamide), or sterates, (e.g., stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate, stearyl stearate); and
a ceramide, optionally formulated at between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more, including any of the ceramides 1-9, and other ceramide derivatives.

In alternative embodiments, these three ingredients are used in various ratios ranging e.g., from 5:1:1 to 1:5:1 to 1:1:5, 4:1:1 to 1:4:1 to 1:1:4, 3:1:1 to 1:3:1 to 1:1:3, 2:1:1 to 1:2:1 to 1:1:2, or any variation thereof.

In alternative embodiments, the total lipid contents used in the topical formulations of the invention (or used to practice a method of the invention) are not critical and can vary over a wide range, even to the upper limit of dissolvability. In alternative embodiments, the total lipid content can range from 0.1% to 60% by weight, or 1% to 20% by weight, or any variation thereof.

In alternative embodiments, topical formulations containing the lipid combinations of the invention are applied to beneficial effect to skin and/or mucus membranes. In alternative embodiments, topical formulations include lipids dispersed or dissolved in a pharmaceutically acceptable carrier, which includes any of the wide variety of vehicles used for application of a medicament to the epidermis. These vehicles are well known in the art. The formulations may assume any of various forms. Examples are lotions, solutions, gels, creams, emollient creams, unguents, skin patches or adhesives, aerosols and sprays.

In alternative embodiments, for many applications, also included are a variety of inactive agents in the formulations to e.g., promote stability, act as a preservative, to promote even spreading of the formulation over the affected area, to improve the odor, and the like. Examples of inactive agents that can be used are surfactants, humectants, wetting agents, emulsifiers, or propellants.

In alternative embodiments, formulations of the invention, or formulations used to practice the methods of the invention, comprise other products, e.g., to enhance barrier function, including urea (optionally formulated at 0.05% up to 40%) and allantoin (optionally formulated at 0.01% up to 2%, plus certain derivatives of allantoin such as aluminium salts, aluminium chlorhydroxyallantoinate (optionally formulated at 0.05% to 2.0%), aluminium dihydroxyallantoinate (optionally formulated at 0.2% to 2%), aluminium chlorhydroxyallantoinate propylene glycol (optionally formulated at 1% to 20%), allantoin N-acetyl dimethionine (optionally formulated at 0.2% to 1.0%), allantoin calcium pantothenate (optionally formulated at 0.1% to 2.0%), allantoin dipanthenol (optionally formulated at 0.1% to 1.0%), allantoin glycyrrhetinate, allantoin polygalacturonate (optionally formulated at 0.1% to 1.0%), allantoin zinc undecylenate, allantoin ethyl p-aminobenzoate (optionally formulated at 0.5% to 5.0%), allantoin silver acetate (optionally formulated at 0.1% to 2.0%), allantoin ascorbate (optionally formulated at 0.1% to 2.0%).

In alternative embodiments, formulations of the invention, or formulations used to practice the methods of the invention (e.g., topical formulations such as gels, lotions, sprays, emollients, skin patches or adhesives, aerosols and the like), comprise anti-inflammatory agents, e.g., nicotinamide (optionally formulated at 0.01% up to 10%).

In alternative embodiments, formulations of the invention, or formulations used to practice the methods of the invention (e.g., topical formulations such as gels, lotions, sprays, emollients, skin patches or adhesives, aerosols and the like), comprise vasodilators, e.g., local vasodilators, e.g. to improve hemoperfusion of the affected areas of the skin. In alternative embodiments, vasodilators include angiotensin converting enzyme inhibitors, such as captopril (optionally CAPOTEN™) (optionally formulated at 0.1% to 5.0%), enalapril (optionally RENITEC™, VASOTEC™) (optionally formulated at 0.1% to 5.0%), lisinopril (optionally PRINIVIL™, TENSOPRIL™, ZESTRIL™) (optionally formulated at 0.1% to 5.0%)), angiotensin receptor type II blockers (e.g., losartan (optionally COZAAR™) (optionally formulated at between about 0.1% to 5.0%), embusartan (optionally formulated at between about 0.1% to 5.0%), fonsartan (optionally formulated at between about 0.1% to 5.0%), pratosartan (optionally formulated at between about 0.1% to 5.0%)), vasodilators that are phosphodiesterase subtype-selective inhibitors (e.g., sildenafil (optionally formulated at 0.1% to 10%), tadalafil (optionally ADCIRCA™ or CIALIS™) (optionally formulated at 0.1% to 10%), and vardenafil (optionally LEVITRA™) (0.1% to 10%)), and direct vasodilators that block K_{ATP} channels (e g, minoxidil (optionally ROGAINE™), optionally formulated at between about 0.1% to 10%; pinacidil (or equivalent cyanoguanidine drug), optionally formulated at between about 0.1% to 10%; and naminidil, optionally formulated at between about 0.1% to 10%).

In alternative embodiments, formulations of the invention, or formulations used to practice the methods of the invention, are made or comprised as described e.g., in U.S. Pat. No. 5,643,899; for example, comprising a cholesterol and an acylceramide a mole ratio of lipid cholesterol to acylceramide from about 0.25:1 to about 5:1); or, comprising: (a) cholesterol, (b) an acylceramide, (c) at least one fatty acid of 12 to 20 carbon atoms in length, the mole ratios of lipids (a):(b):(c) being within the ranges (0.25-5):(1-3):(1.5-3.5); or, comprising: (a) cholesterol, (b) a ceramide, (c) an essential fatty acid, and (d) a nonessential fatty acid of 12 to 20 carbon atoms in length, the mole ratios of lipids (a):(b):(c):(d) being within the ranges (2-5):(1-3):(1-3):(1.5-3.5); or 3:1:1:1, 2:1:1:1, 2:2:1:1, 1:1:1:2, and 1:1:1:3; or, comprising: (a) cholesterol, (b) a glycoceramide, (c) at least one fatty acid of 12 to 20 carbon atoms in length, the mole ratios of lipids (a):(b):(c) being within the ranges (0.25-5):(1-3):(1.5-3.5).

In alternative embodiments, formulations of the invention, or formulations used to practice the methods of the invention, are made or comprised as described e.g., in U.S. Pat. Nos. 7,550,135; 6,824,785 (e.g., comprising an aqueous formulation of at least three lipids in a non-crystalline phase lamellar array which adopt a crystalline lamellar phase upon application to mammalian skin, and the at least three lipids comprise a ceramide, a saturated fatty acid and cholesterol; and the composition comprises a brain ceramide, or a palmitic acid, and/or cholesterol in ratios by mole of from 1-5:1-5:1-5, respectively); U.S. Pat. Nos. 6,756,520; 6,749,860.

In alternative embodiments, the invention provides physiological balanced formulations, e.g., lotions, creams (e.g., hand/foot creams), gels and the like, that can:
Enhance the hydration, flexibility and biomechanical properties of the skin,
reduce callus formation secondary to poorly distributed pressure gradients
reduce hyperkeratosis and metabolic load on tissue and demand on capillary beds,
improve resistance to stressors, including keratinocytes and capillary beds from micro-traumas, and/or
minimize chronic use topical glucocorticoids and other agents that may impair tissue integrity.

Prophylactic and/or Ameliorative Treatment for Dermatological Toxicity and HFSR

In alternative embodiments compositions and methods of the invention provide a prophylactic and/or ameliorative treatment for dermatological toxicity and HFSR (also known as palmar-plantar erythrodysesthesia), a significant dose-limiting toxicity associated with administration of an oral oncolytic, a targeted kinase inhibitor, a Multi Kinase Inhibitor (MKI), an Epidermal Growth Factor Receptor Inhibitor (EGFRI), a cancer drug, or a combination thereof, e.g., where MKIs are used to treat solid tumors. In alternative embodiments compositions and methods of the invention are administered before beginning an oral oncolytic, a targeted kinase inhibitor, a Multi Kinase Inhibitor (MKI), an Epidermal Growth Factor Receptor Inhibitor (EGFRI), a cancer drug, or a combination thereof, therapy, or when initiating the therapy, but in alternative embodiments no later than 2 to 4 weeks after initiating therapy (e.g., a therapy comprising administration of an oral oncolytic, a targeted kinase inhibitor, a Multi Kinase Inhibitor (MKI), an Epidermal Growth Factor Receptor Inhibitor (EGFRI), a cancer drug, or a combination thereof), because dermatological toxicity or HFSR develops 2 to 4 weeks after initiating the therapy.

MKI therapy is characterized by painful lesions on the skin that are tender and scaling, ringed by erythemateous skin. The lesions are located primarily in palmar-plantar sites prone to pressure or mechanical stress, and subungual splinter hemorrhages and other lesions of the periungual regions may also present. The skin lesions and blisters develop near areas of thickened “hyperkeratotic” skin, and are accompanied by inflammatory immune infiltrates.

While the invention is not limited by any particular mechanism of action, the incidence of dermatological toxicity or HFSR may be related to the cumulative dose of the oral oncolytic, targeted kinase inhibitor, MKI, and the like, and can be resolved by practicing the compositions and methods of the invention without reducing or interrupting the drug therapy (reducing or interrupting the drug therapy results in significant tachyphylaxis upon reinstituting therapy).

“Hand Foot Syndrome” associated with conventional cytotoxic therapy and other dermatological toxicity that accompanies another class of targeted kinase inhibitors that blocks EGF signaling, is differentiated from HFSR both clinically and histopathologically. HFSR remains a clinical diagnosis with no specific diagnostic testing recommended for confirmation. While tissue biopsy may be useful in the research setting to gain additional insight into the mechanism and pathophysiology of HFSR, it is not appropriate in routine clinical practice.

While the invention is not limited by any particular mechanism of action, the MKIs can precipitate dermatological toxicity or HFSR due to simultaneous inhibition of both VEGF-R and PDRGF-R, and perhaps other receptor tyrosine kinases. Neither anti-VEGF mAbs (bevacizumab) nor PDGF-R inhibitors (e.g., imatinib) lead to HFSR, but bevacizumab combined with sorafenib exacerbates skin toxicity. Thus, in one embodiment, compositions and methods of the invention are used before and/or with treatment protocols comprising bevacizumab combined with sorafenib.

### Methods of Administration

This invention provides compositions (e.g., for use in the exemplary combinations of drugs of the invention), e.g., pharmaceutical compositions, preparations and kits, that can be administered by several routes, including intravenous, topical and oral, or combinations thereof.

For example, one embodiment comprises a product of manufacture comprising a pharmaceutical composition or a formulation, a blister package, a lidded blister or a blister card or packet, a clamshell, a tray or a shrink wrap, or a kit, comprising: (i) (a) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof, and, (b) a pharmaceutical composition or formulation comprising a nicotinamide, niacinamide or nicotinic acid amide, or an equivalent thereof. This exemplary product of manufacture can further comprising a urea (optionally as a urea cream) and/or a keratolytic. In alternative embodiments, although all ingredients can be in one blister package, a lidded blister or a blister card or packet, a clamshell, a tray or a shrink wrap, or a kit, the physiologically balanced lipid formulation and urea and/or a keratolytic are formulated for topical application, and the nicotinamide is either formulated for oral or topical application. Each ingredient can be either separately packaged, or can be formulated as one unit dose, e.g., as one tube (e.g., with gel, lotion etc.), ampoule, blister packette and the like.

In alternative embodiments, this invention provides forms of compositions, preparations and kits that can be administered by inhalation, infusion or injection, (e.g., intraperitoneal, intramuscular, subcutaneous, intra-aural, intra-articular, intra-mammary, etc.), topical application (e.g., on areas, such as eyes, ears, skin or on afflictions such as wounds, burns, etc.), and by absorption through epithelial or mucocutaneous linings (e.g. vaginal and other epithelial linings, gastrointestinal mucosa, etc.). Methods are known for making compositions, preparations and kits containing the present components that are suitable for each of these methods of administration as well as other methods of administration that are know in the art.

In alternative embodiments, this invention provides compositions, preparations and kits in liquid forms that can be administered orally. The compositions, preparations and kits can be also prepared as capsules, gels, geltabs, tablets, powders, sprays, aerosols, pellets (e.g. for animal consumption), suppositories, lotions, patches or adhesives (e.g., for the skin), or creams and ointments. The compositions, preparations and kits can be also prepared as physiological solutions suitable for I.V. administration or other parenteral administration.

In one aspect, a multi-ingredient kit of the invention comprises (contains) two or more ingredients in approximately equal amounts. An amount may be determined, e.g. by mass or by weight or by molar amount. In another aspect, a multi-ingredient kit may contain two or more ingredients in unequal amounts. In another aspect, a multi-ingredient kit may contain two or more ingredients in approximately equal amounts as well as one or more ingredients that are not in unequal amounts.

Thus, in alternative embodiments, a multi-ingredient kit may contain two or more ingredients in approximately equimolar amounts. In another embodiment, a multi-ingredient kit may contain two or more ingredients that are not in equimolar amounts. In another aspect, a multi-ingredient kit may contain two or more ingredients that are in approximately equimolar amounts as well as one or more ingredients that are not in equimolar amounts.

In another embodiment, said multi-ingredient kit may contain two or more ingredients that are admixed. In another aspect, said multi-ingredient kit may contain two or more ingredients that are not admixed. In another aspect, said multi-ingredient kit may contain two or more ingredients that are partially admixed. In another aspect, said multi-ingredient kit may contain two or more ingredients that are at least partially admixed, as well as one or more ingredients that are not admixed. An ingredient in a multi-ingredient kit may be liquid forms that can be administered orally.

In another embodiment, an ingredient in a multi-ingredient kit may also be in delivery forms such as capsules, tablets, powders, sprays, aerosols, pellets (e.g. for animal consumption), suppositories, or creams and ointments. An ingredient in a multi-ingredient kit may also be in delivery forms such as physiological solutions suitable for I.V. administration or other parenteral administration.

In another embodiment, the ingredients in a multi-ingredient kit may be separated by physically compartmentalization (e.g. in separate compartments that are part of said kit, where said kit is a multi-compartment kit). Thus, for example, it is provided that the ingredients may be admixed or not admixed. For example, a single pill or capsule may contain more than one key ingredient (e.g. an ACEi, an oral oncolytic, a targeted kinase inhibitor, a Multi Kinase Inhibitor (MKI), an Epidermal Growth Factor Receptor Inhibitor (EGFRI), a cancer drug, or a combination thereof). Alternatively, separate compartments, as may be found in a “blister pack” type of packaging, may contain different ingredients.

### Packaging

The invention provides compositions, including preparations, formulations and/or kits, comprising combinations of ingredients, as described herein. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the pharmaceuticals of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one aspect, a blister packs also comprise a method of packaging where the compositions comprising combinations of ingredients of the invention are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two components (e.g., is a multi-ingredient combination of drugs of the invention): a thermoformed “blister” which houses the product (e.g., a pharmaceutical combination of the invention), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

### Blister Packaging

In alternative embodiments, combinations of the invention can comprise the packaging of the therapeutic drug combinations of the invention, alone or in combination, as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of drugs designed to dissolve immediately in the mouth of a patient. This exemplary process comprises having the drug combinations of the invention prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminum (e.g., alufoil) lidding material. In one aspect, hermetically-sealed high barrier aluminum (e.g., alufoil) laminates are used. In one aspect, any of the invention's products of manufacture, including kits or blister packs, use foil laminations and strip packs, stick packs, sachets and pouches, peelable and non-peelable laminations combining foil, paper, and film for high barrier packaging.

In alternative embodiments, any of the invention's products of manufacture, including kits or blister packs, include memory aids to help remind patients when and how to take the drug. This safeguards the drug's efficacy by protecting each pill until it's taken; gives the product or kit portability, makes it easy to take a dose anytime or anywhere.

In alternative embodiments, the invention provides a dermatological toxicity or an HFSR regimen as a starter kit plus a maintenance kit. The starter kit can comprise e.g., video/training in addition to e.g., a combination of the invention, e.g., a dose escalation of e.g., a multi-kinase-inhibitor, ACEi and lotion; or an oral oncolytic, a targeted kinase inhibitor, a targeted Multi Kinase Inhibitor (MKI), ACEi and lotion; and the like. In alternative embodiments, the invention provides maintenance kits comprising, e.g., ACEi and lotion. Both kits may include smart packaging to enabling tacking of adherence.

In alternative embodiments, compositions of the invention, e.g., gels, creams, lotions, sprays, patches, adhesives and the like, comprise physiologically balanced lipids; which in some embodiments can function as a moisturizers to improve the mechanical properties of the skin that prevent koebner-related (external mechanical) injury to the skin/capillary; and in some embodiments (noting the invention is not limited by any particular mechanism of action) reduce metabolic load which will help keep tissue oxygen use/supply in balance and avoid damage. In alternative embodiments, additional components are added to the compositions of the invention, e.g., gels, creams, lotions, sprays, patches, adhesives and the like, such as urea, anti-oxidants and active drugs such as glucocorticoids and vasodilators.

In alternative embodiments, the invention provides compliance kits; which can be used to solve several problems that make patient adherence of complex regimens, especially topical therapy, problematic. In alternative embodiments, compliance kits of the invention are designed to solve the various patient adherence problems including: (1) eliminate difficulty in assembling components: the patient does not have to go to store, figure out which products to buy and then products home, e.g., every month (2) eliminate self-pay/co-pay for the components that discourage usage (3) encourage adherence with instructional video/packaging that explain why good idea (4) encourage adherence with phone calls from specialty pharmacy to answer question and review usage (5) encourage adherence with smart packaging/embedded chips to track usage and alert specialty pharmacy/care givers/physician to non-adherence so corrective measure can be taken.

In alternative embodiments, starter, maintenance and/or compliance kits of the invention also comprise full-moisture barrier gloves/socks, such as a latex lined glove/socks, that would improve moisture content of skin/slow metabolic activity to improve mechanical properties of skin and reduce metabolic load.

A number of aspects of the invention have been described. Nevertheless, it will be understood that various modifications may be made without departing from the spirit and scope of the invention. Accordingly, other aspects are within the scope of the following claims.

1. A product of manufacture comprising a pharmaceutical composition or a formulation, a blister package, a lidded blister or a blister card or packet, a clamshell, a tray or a shrink wrap, or a kit, comprising:
(a) (i) a pharmaceutical composition or formulation comprising a nicotinamide, niacinamide or nicotinic acid amide, or an equivalent thereof; and
(ii) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof;
(b) the product of manufacture of (a), wherein the composition or formulation comprises an ampoule, a gel, a lotion, a cream, an emollient, a skin patch or adhesive, aerosol or a spray for topical application;
(c) the product of manufacture of (a) or (b), wherein:
(i) the formulation has about 4%, 5%, 6%, 7%, 8% or more, or about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: nicotinamide, niacinamide or nicotinic acid amide, or an equivalent thereof;
(ii) the formulation has about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
(ii) the formulation has about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids,
(iii) the formulation has about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: ceramide or acylceramide;
(d) the product of manufacture of any of (a) to (c), further comprising instructions for use;
(e) the product of manufacture of any of (a) to (d), further comprising: a urea or a keratolytic; an anti-oxidant; a retinoid; a glucocorticoid; a vasodilator; a diprobase; a formulation of white soft paraffin, a cetomacrogol and a cetostearyl alcohol; a tadalafil; a direct arterial vasodilator; or any combination thereof;
(f) the product of manufacture of any of (a) to (e), further comprising a pharmaceutical composition or formulation comprising a vasodilator.

2. A product of manufacture comprising a pharmaceutical composition or a formulation, a blister package, a lidded blister or a blister card or packet, a clamshell, a tray or a shrink wrap, or a kit, comprising:
(a) (i) a pharmaceutical composition or formulation comprising a vasodilator; and
(ii) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof,
wherein optionally the fatty acid; ceramide or acylceramide, sphingosine-fatty acid, or equivalent thereof; and cholesterol, a (3β)-cholest-5-en-3-ol, or equivalent thereof, are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2;
(b) the product of manufacture of (a), wherein the composition or formulation comprises an ampoule, a gel, a lotion, a cream, an emollient, a skin patch or adhesive, aerosol or a spray for topical application,
wherein optionally the ampoule, gel, lotion, cream, emollient, skin patch or adhesive, aerosol or spray is packaged and/or formulated as a single unit dosage, for example, one (a single) dosage of a gel, lotion, cream or emollient is packaged in its own (is contained in a single (one)) tube, ampoule or packette
(c) the product of manufacture of (a) or (b), wherein:
(i) the vasodilator comprises: an Angiotensin Converting Enzyme inhibitor (ACEi), an angiotensin receptor type II blocker, an embusartan, a fonsartan, a pratosartan, a phosphodiesterase subtype-selective inhibitor, a tadalafil (optionally ADCIRCA™ or CIALIS™), a vardenafil (optionally LEVITRA™) (optionally LEVITRA™), a direct vasodilator that blocks a K_{ATP} channel, a pinacidil, a naminidil, or a combination thereof;
wherein optionally:
the Angiotensin Converting Enzyme inhibitor (ACEi) is
a captopril (optionally CAPOTEN™), optionally formulated at between about 0.1% to 5.0%, or between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
an enalapril (optionally RENITEC™, VASOTEC™), optionally formulated at between about 0.1% to 5.0%, or between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
a lisinopril (optionally PRINIVIL™, TENSOPRIL™, ZESTRIL™), optionally formulated at between about 0.1% to 5.0%, or between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more, or
the Angiotensin Converting Enzyme inhibitor (ACEi) is benazepril (optionally LOTENSIN™), captopril (optionally CAPOTEN™), cilazapril (optionally INHIBACE™, ZAPRIL™, VASCACE™), enalapril (optionally RENITEC™, VASOTEC™), enalaprilat, fosinopril (optionally MONOPRIL™), imidapril, lisinopril (optionally PRINIVIL™, TENSOPRIL™, ZESTRIL™), moexipril (optionally UNIVASC™, PERDIXS™), perindopril (optionally COVERSYL™, ACEON™), quinapril (optionally ACCUPRIL™) ramipril (optionally ALTACE™, PRILACE™), trandolapril (optionally MAVIK™) or a combination thereof;
the angiotensin receptor type II blockers is a losartan (optionally COZAAR™) optionally formulated at (0.1% to 5.0%), an embusartan (optionally formulated at 0.1% to 5.0%), a fonsartan (optionally formulated at 0.1% to 5.0%), a pratosartan (optionally formulated at 0.1% to 5.0%);
the phosphodiesterase subtype-selective inhibitor is
a sildenafil, optionally formulated at 0.1% to 10%, or between about 1.0% to 10%, or about 1%, 2%, 3%, 4%, 5%, 6%, 7%, 8%, 9%, 10% or more; or between about 2% to 8%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
a tadalafil (optionally CIALIS™), optionally formulated at about 0.1% to 10%, or between about 1.0% to 10%, or about 1%, 2%, 3%, 4%, 5%, 6%, 7%, 8%, 9%, 10% or more; or between about 2% to 8%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more, or
a vardenafil (optionally LEVITRA™), optionally formulated at 0.1% to 10%, or between about 1.0% to 10%, or about 1%, 2%, 3%, 4%, 5%, 6%, 7%, 8%, 9%, 10% or more; or between about 2% to 8%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more;
the direct vasodilator that blocks K_{ATP} channels is a minoxidil (optionally ROGAINE™) (optionally formulated at about 0.1% to 10%);
the pinacidil is optionally formulated at between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more;
the naminidil is optionally formulated at between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more;
(ii) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
(iii) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids, wherein optionally the fatty acids comprise: at least two essential fatty acids, or alpha-linolenate and/or linoleate; a lecithin; an oleate, or an oleic acid, oleyl oleate or oleyl stearate; a palmitate, or a palmitate, palmitamine or palmitamide; a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate,
(iv) the formulation has about 0.1% to 10% (or about 1%, 2%, 3%, 4%, 5%, 6%, 7%, 8%, 9%, 10% or more) ceramide or acylceramide, wherein optionally the ceramide or acylceramide comprises a ceramide 1-9, or a ceramide derivative;
(d) the product of manufacture of any of (a) to (c), further comprising instructions for use; or
(e) the product of manufacture of any of (a) to (d), further comprising: a urea (optionally as a urea cream) and/or a keratolytic, a petrolatum or an occlusive; a glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase, or a combination thereof; a formulation of white soft paraffin, a cetomacrogol and a cetostearyl alcohol; a tadalafil (optionally CIALIS™) (optionally to improve perfusion of distal vascular beds); a direct arterial vasodilator (optionally a minoxidil (optionally ROGAINE™)); or any combination thereof.

3. A product of manufacture comprising a pharmaceutical composition or a formulation, a blister package, a lidded blister or a blister card or packet, a clamshell, a tray or a shrink wrap, or a kit, comprising:
(a) (i) a pharmaceutical composition or formulation comprising an angiotensin converting enzyme inhibitor (ACEi), or an angiotensin receptor type II blocker; and
(ii) a composition comprising a physiologically balanced lipid formulation, wherein the formulation comprises: (1) a least one fatty acid, (2) at least one ceramide or acylceramide, a sphingosine-fatty acid, or equivalent thereof, and (3) at least one cholesterol, a (3β)-cholest-5-en-3-ol, or equivalent thereof,
wherein optionally the fatty acid; ceramide or acylceramide, sphingosine-fatty acid, or equivalent thereof; and cholesterol, a (3β)-cholest-5-en-3-ol, or equivalent thereof, are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2;
(b) the product of manufacture of (a), wherein the composition or formulation comprises an ampoule, a gel, a lotion, a cream, an emollient, a skin patch or adhesive, aerosol or a spray for topical application,
wherein optionally the ampoule, gel, lotion, cream, emollient, skin patch or adhesive, aerosol or spray is packaged and/or formulated as a single unit dosage, for example, one (a single) dosage of a gel, lotion, cream or emollient is packaged in its own (is contained in a single (one)) tube, ampoule or packette;
(c) the product of manufacture of (a) or (b), wherein:
(i) the angiotensin converting enzyme inhibitor (ACEi) comprises a captopril (optionally CAPOTEN™) (optionally formulated at about 6.25 mg to 50 mg), an enalapril (optionally RENITEC™, VASOTEC™) (optionally formulated at about 1 mg to 100 mg), a lisinopril (optionally PRINIVIL™, TENSOPRIL™, ZESTRIL™) (optionally formulated at about 2.5 mg to 160 mg); or
the angiotensin receptor type II blocker comprises a losartan (optionally COZAAR™) (optionally formulated at about 6.25 mg to 200 mg), an embusartan (optionally formulated for administration at about 0.1 to 30 mg/kg), a fonsartan (optionally formulated for administration at about 0.1 to 30 mg/kg), pratosartan (optionally formulated for administration at about 10 to 320 mg/day);
(ii) the formulation has between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: cholesterol, (3β)-cholest-5-en-3-ol, or equivalent;
(iii) the formulation has about between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: fatty acids, wherein optionally the fatty acids comprise: at least two essential fatty acids, or alpha-linolenate and/or linoleate; a lecithin; an oleate, or an oleic acid, oleyl oleate or oleyl stearate; a palmitate, or a palmitate, palmitamine or palmitamide); a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate)
(iv) the formulation has about between about 1.0% to 10%, or about 1%, 2%, 3%, 4%, 5%, 6%, 7%, 8%, 9%, 10% or more; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more: ceramide or acylceramide, wherein optionally the ceramide or acylceramide comprises a ceramide 1-9, or a ceramide or acylceramide derivative;
(d) the product of manufacture of any of (a) to (c), further comprising instructions for use; or
(e) the product of manufacture of any of (a) to (d), further comprising: a urea (optionally as a urea cream) and/or a keratolytic, a petrolatum or an occlusive; a glycerol or a humectant; citric acid or a pH buffer; one or more tocopherol(s) or an anti-oxidant; a retinoid (optionally a tazarotene); a glucocorticoid (optionally clobetasol propionate); a vasodilator; a diprobase, or a combination thereof; a formulation of white soft paraffin, a cetomacrogol and a cetostearyl alcohol; a tadalafil (optionally CIALIS™) (optionally to improve perfusion of distal vascular beds); a direct arterial vasodilator (optionally a minoxidil (optionally ROGAINE™)); or any combination thereof.

4-18. (canceled)

19. The product of manufacture of claim 1, wherein the:
(1) a least one fatty acid,
(2) at least one ceramide or acylceramide, a sphingosine-fatty acid, or equivalent thereof, and
(3) at least one cholesterol, a (3β)-cholest-5-en-3-ol, or equivalent thereof,
are used in ratios ranging from about 5:1:1 to 1:5:1 to 1:1:5, or from about 4:1:1 to 1:4:1 to 1:1:4, or from about 3:1:1 to 1:3:1 to 1:1:3, or from about 2:1:1 to 1:2:1 to 1:1:2.

20. The product of manufacture of claim 1, wherein the ampoule, gel, lotion, cream, emollient, skin patch or adhesive, aerosol or spray is packaged and/or formulated as a single unit dosage, for example, one or a single dosage of a gel, lotion, cream or emollient is packaged in its own or is contained in a single or one tube, ampoule or packette.

21. The product of manufacture of claim 1, wherein the fatty acids comprise: at least two essential fatty acids, or an alpha-linolenate and/or a linoleate; a lecithin; an oleate, or an oleic acid, oleyl oleate or oleyl stearate; a palmitate, or a palmitate, palmitamine or palmitamide; a stearate, or a stearamide, stearamine, stearamine oxide, stearic acid, stearic hydrazide, stearone, stearoxy trimethylsilane, stearoyl lactylate, stearyl acetate, stearyl alcohol, stearamine oxide, stearyl betaine, tearyl caprylate, stearyl citrate, stearyl dimethylamine, stearyl glycyrrhetinate, stearyl heptanoate, stearyl imidazoline, stearyl octanoate or a stearyl stearate.

22. The product of manufacture of claim 1, wherein the ceramide or acylceramide comprises a ceramide 1-9, or comprises a ceramide or acylceramide derivative.

23. The product of manufacture of claim 1, wherein: the urea is a urea cream; the retinoid comprises a tazarotene; the glucocorticoid comprises a clobetasol propionate; or, the direct arterial vasodilator comprises a minoxidil.

24. The product of manufacture of claim 1, wherein the vasodilator comprises: an Angiotensin Converting Enzyme inhibitor (ACEi), an angiotensin receptor type II blocker, an embusartan, a fonsartan, a pratosartan, a phosphodiesterase subtype-selective inhibitor, a tadalafil, a vardenafil, a direct vasodilator that blocks a K_{ATP} channel, a pinacidil, a naminidil, or a combination thereof.

25. The product of manufacture of claim 1, wherein the Angiotensin Converting Enzyme inhibitor (ACEi) comprises:
a captopril, optionally formulated at between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more;
an enalapril, optionally formulated at between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more; or
a lisinopril, optionally formulated at 0.1% to 5.0%; between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more.

26. The product of manufacture of claim 1, wherein the Angiotensin Converting Enzyme inhibitor (ACEi) comprises: a benazepril, a captopril, a cilazapril, an enalapril, an enalaprilat, a fosinopril, an imidapril, a lisinopril, a moexipril, a perindopril, a quinapril, a ramipril, a trandolapril, or a combination thereof.

27. The product of manufacture of claim 1, wherein the angiotensin receptor type II blocker comprises:
a losartan (optionally COZAAR™), optionally formulated at between about 0.1% to 5.0%, or between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
an embusartan, optionally formulated at between about 0.1% to 5.0%, between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
a fonsartan, optionally formulated at between about 0.1% to 5.0%, between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more, or
a pratosartan, optionally formulated at between about 0.1% to 5.0%, or between about 1.0% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more.

28. The product of manufacture of claim 1, wherein the phosphodiesterase subtype-selective inhibitor comprises:
a sildenafil, optionally formulated at 0.1% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
a tadalafil (optionally ADCIRCA™ or CIALIS™), optionally formulated at about 0.1% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more, or
a vardenafil (optionally LEVITRA™), optionally formulated at 0.1% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more.

29. The product of manufacture of claim 1, wherein the direct vasodilator that blocks K_{ATP} channels comprises:
a minoxidil, optionally formulated at about 0.1% to 10%; or between about 2% to 8%; or between about 0.1% to 10%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more,
a pinacidil, optionally formulated at about 0.1% to 10%; or between about 2% to 8%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more, or
a naminidi, optionally formulated at about 0.1% to 10%; or between about 2% to 8%; or about 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or 10% or more.

30. The product of manufacture of claim 1, further comprising an oral oncolytic, a targeted kinase inhibitor, a Multi Kinase Inhibitor (MKI), an Epidermal Growth Factor Receptor Inhibitor (EGFRI), a cancer drug, or a combination thereof.

31. The product of manufacture of claim 30, wherein the MKI comprises:
a sorafenib or a sunitinib, or
the MKI is formulated for a dose-escalation protocol, optionally comprising a 100 mg dose (⅛^{th} the approved dose) on days 1 to 3, 200 mg (¼^{th} the approved dose) on days 4 to 6, 200 mg twice per day (½ the approved dose) on days 7 to 9, and 400 mg twice per day on day 10 and beyond.

32. The product of manufacture of claim 30, wherein the oral oncolytic comprises a capcitabine, or
wherein the oral oncolytic is used at a dosage of about 150 mg, 300 mg, 500 mg, 1000 mg, 1500 mg, 2000 mg, 2500 mg, 3000 mg, 3500 mg, 4000 mg, 4500 mg, or 5000 mg.

33. The product of manufacture of claim 30, wherein the EGFRI comprises an erlotinib, or
the EGFRI is used at a dosage of about 25, 50, 75, 100, 125, 150, 175, 200, 225, or 250 mg, or between about 20 to 300 mg.

34. The product of manufacture of claim 30, wherein the cancer drug comprises a gemcitabine or a docetaxel.

35. The product of manufacture of claim 1, further comprising an angiotensin converting enzyme inhibitor (ACEi), or an angiotensin receptor type II blocker;
and optionally:
(a) the angiotensin converting enzyme inhibitor (ACEi) comprises:
a captopril, optionally formulated at about 6.25 mg to 50 mg,
an enalapril, optionally formulated at about 1 mg to 100 mg,
a lisinopril, optionally formulated at about 2.5 mg to 160 mg; or
(b) the angiotensin receptor type II blocker comprises:
a losartan, optionally COZAAR™, optionally formulated at about 6.25 mg to 200 mg,
an embusartan, optionally formulated for administration at about 0.1 to 30 mg/kg,
a fonsartan, optionally formulated for administration at about 0.1 to 30 mg/kg, or
a pratosartan, optionally formulated for administration at about 10 to 320 mg/day.
